(19) United States
(12) Patent Application Publication
(10) Pub. No.: US 2023/0404424 A1
(43) Pub. Date: Dec. 21, 2023

(54) 3-AXIS MAGNETIC POSITION SYSTEM FOR MINIMALLY INVASIVE SURGICAL INSTRUMENT, SYSTEMS AND METHODS THEREOF

(71) Applicant: Analog Devices International Unlimited Company, Limerick (IE)

(72) Inventors: Yogesh Jayaraman SHARMA, Santa Clara, CA (US); Christopher W. HYDE, Hollis, NH (US); Brendan CRONIN, Cork (IE); Jochen SCHMITT, Biedenkopf (DE)

(21) Appl. No.: 18/461,062

(22) Filed: Sep. 5, 2023

Related U.S. Application Data
(63) Continuation of application No. 15/995,130, filed on Jun. 1, 2018.
(60) Provisional application No. 62/513,904, filed on Jun. 1, 2017.

(51) Int. Cl. A61B 5/06; A61B 5/00; G01R 33/02; A61B 5/055; G01R 33/09

(52) U.S. Cl. CPC A61B 5/062; A61B 5/6852; G01R 33/0206; A61B 5/055; G01R 33/093; A61M 25/0127

(57) ABSTRACT

A minimally invasive surgical instrument using 3-axis magnetic positioning, system and methods thereof. This invention describes two key ideas that enable the development of a magnetic position system based on integrated anisotropic magnetoresistive (AMR) magnetic field sensors. This achieves the resolution, power and area targets necessary to integrate 3 axes anisotropic magnetoresistance (AMR) sensors along with the Analog Front End integrated circuit (IC) in a 4 mm by 350 um integrated solution for catheter applications. The stringent area and power dissipation requirements are met by development through both system level solutions for higher field strengths and a minimally necessary Analog Front End (AFE) to meet the 1 mm rms resolution requirement in the power dissipation and area budget.

### CROSS-REFERENCE TO RELATED APPLICATION

This application is related and claims priority to U.S. Provisional Application No. 62/513,904 entitled, “3-Axis Magnetic Position System for Minimally Invasive Surgical Instrument, Systems and Methods Thereof” filed on Jun. 1, 2017, which is hereby incorporated by reference in its entirety.

### FIELD OF THE DISCLOSURE

The present disclosure relates to magnetic positioning systems, primarily in situ. More specifically, this disclosure describes apparatus and techniques relating to lower power, smaller scale and higher resolution positioning catheters.

### BACKGROUND

Catheters are tubular medical devices that may be inserted into a body vessel, cavity or duct, and manipulated utilizing a portion that extends out of the body. Typically, catheters are relatively thin and flexible to facilitate advancement/retraction along non-linear paths. Catheters may be employed for a wide variety of purposes, including the internal bodily positioning of diagnostic and/or therapeutic devices. For example, catheters may be employed to position internal imaging devices, deploy implantable devices (e.g., stents, stent grafts, vena cava filters), and/or deliver energy (e.g., ablation catheters).

Accurate lumen positioning is of paramount importance. However, it has long since been desired in the art to achieve high resolution positioning while keeping within certain limiting parameters, specifications and medical standards. The inventors of the present disclosure have recognized that this can be achieved using a combination of novel techniques, systems and apparatus.

This overview is intended to provide an overview of subject matter of the present patent application. It is not intended to provide an exclusive or exhaustive explanation of the invention. Further limitations and disadvantages of conventional and traditional approaches will become apparent to one of skill in the art, through comparison of such systems with some aspects of the present invention as set forth in the remainder of the present application with reference to the drawings.

### SUMMARY OF THE DISCLOSURE

This invention describes two key ideas that enable the development of a magnetic position system based on integrated anisotropic magnetoresistive (AMR) magnetic field sensors. This achieves the resolution, power and area targets necessary to integrate 3 axes anisotropic magnetoresistance (AMR) sensors along with the Analog Front End IC in a 4 mm by 350 um integrated solution for catheter applications. The stringent area and power dissipation requirements are met by development through both system level solutions for higher field strengths and a minimally necessary Analog Front End (AFE) to meet the 1 mm rms resolution requirement in the power dissipation and area budget.

Those in the art will appreciate that the present disclosure is particularly useful in minimally invasive instruments, such as, catheters, diagnostic pill-scopes, ENT surgical tools, Orthopedic instruments, neurologic probes and instruments, needle biopsy, and more. But the present disclosure may be used in other applications as well, e.g., driver/pilot head position sensing. As such, any application of the positioning system is beyond the inventors' intended scope.

According to one aspect, the present disclosure is a 3-axis positioning apparatus comprising a first die associated with measuring a first axis orientation and/or position, a second die associated with measuring a second and third axes orientation and/or position, and a third die with an analog front end disposed thereon.

According to one or more aspects, the a 3-axis positioning apparatus further comprises a full bridge using magnetoresistive elements. According to another aspect, the a 3-axis positioning apparatus further comprises a half bridge utilizing magnetoresistive elements and another half bridge utilizing poly resistor elements, which the latter can be disposed with the analog front end.

According to one or more aspects, the a 3-axis positioning apparatus further comprises a voltage divider used to measure the common mode voltage between Vdd and GND to increase gain of differential signals and decrease gain of interference signals associated with power supply.

According to one aspect, the invention disclosure is a system and method for increasing resolution while decreasing maximum field exposure. According to one or more aspects, the system and method comprises moving one or more field generators further away from subject/patient, while increasing magnetic field flux by increasing current to said one or more field generators.

The drawings show exemplary magnetic positioning system for minimally invasive surgery, circuits and configurations. Variations of these circuits, for example, changing the positions of, adding, or removing certain elements from the circuits are not beyond the scope of the present invention. The illustrated smoke detectors, configurations, and complementary devices are intended to be complementary to the support found in the detailed description.

### BRIEF DESCRIPTION OF THE DRAWINGS

For a fuller understanding of the nature and advantages of the present invention, reference is made to the following detailed description of preferred embodiments and in connection with the accompanying drawings, in which:

FIG. 1A shows an exemplary positioning instrument with 3 dies disposed within the lumen, in accordance with some embodiments of the disclosure provided herein;

FIG. 1B depict an exemplary isometric view and cross-sectional views of a positioning instrument with 3 dies disposed within the lumen, in accordance with some embodiments of the disclosure provided herein;

FIG. 2 shows an exemplary positioning circuit and architecture, in accordance with some embodiments of the disclosure provided herein;

FIG. 3A graphs maximum exposure limits of magnetic flux density, in accordance with some embodiments of the disclosure provided herein;

FIG. 3B graphs maximum exposure limits of magnetic flux density in practice, in accordance with some embodiments of the disclosure provided herein;

FIG. 4 illustrates an exemplary configuration of field generating coils, in accordance with some embodiments of the disclosure provided herein;

FIG. 5 depicts an exemplary chart demonstrating distal field strengths at two different field generator positions, in accordance with some embodiments of the disclosure provided herein;

FIG. 6 is an exemplary full anisotropic magnetoresistance (AMR) bridge, in accordance with some embodiments of the disclosure provided herein;

FIG. 7 demonstrates an exemplary anisotropic magnetoresistance (AMR) bridge and AFE, in accordance with some embodiments of the disclosure provided herein;

FIG. 8 demonstrates an exemplary anisotropic magnetoresistance (AMR) half bridge with electrical communication with a poly res half bridge disposed on the AFE, in accordance with some embodiments of the disclosure provided herein; and,

FIG. 9 illustrates an exemplary an exemplary anisotropic magnetoresistance (AMR) half bridge with electrical communication with a poly res half bridge disposed on the analog front-end (AFE) and an additional pin to output common mode catheter voltage, in accordance with some embodiments of the disclosure provided herein.

### DETAILED DESCRIPTION

The present disclosure relates to magnetic positioning systems, primarily in situ. More specifically, this disclosure describes apparatus and techniques relating to lower power, smaller scale and higher resolution positioning catheters.

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure are set forth in the proceeding in view of the drawings where applicable.

In one or more embodiments, a magnetic sensor is used to determine its position while being exposed to an external magnetic field. Desirable specifications to be used in as a medical device comprise field strength range, resolution, field frequency range, sampling frequency, minimum sensitivity, package length and diameter, number of axes, operating temperature range, number of wires, and power consumption.

The inventors of the present disclosure accomplish these by methodology and circuit architecture. The former comprises reducing the maximum magnetic exposure of a patient by positioning the field generators further away from the patient. This increases the gap and ability to increase field generating current. The circuit architecture addresses resolution, field frequency range, sampling frequency, minimum sensitivity, package length and diameter, number of axes, operating temperature range, number of wires, and power consumption.

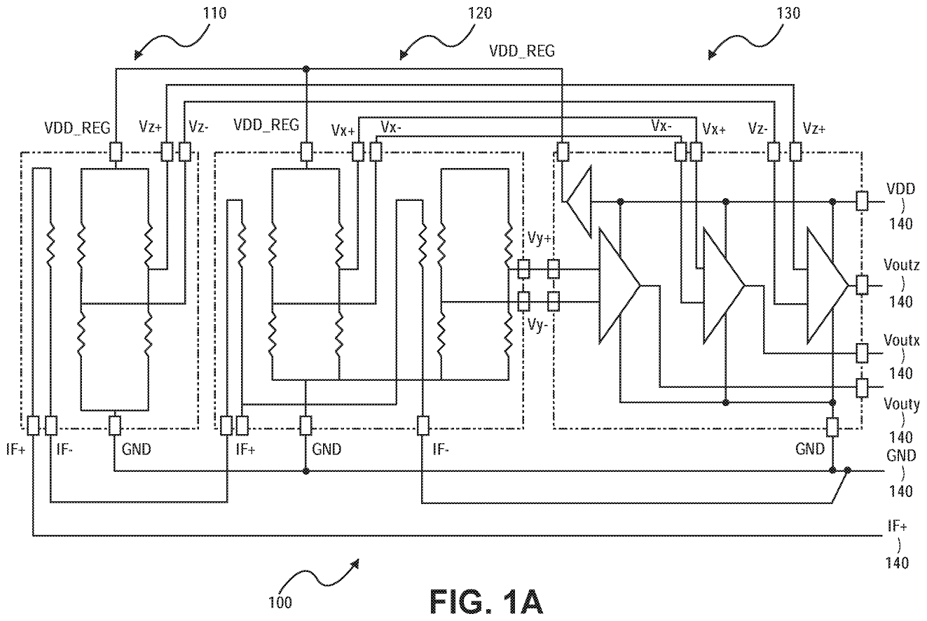

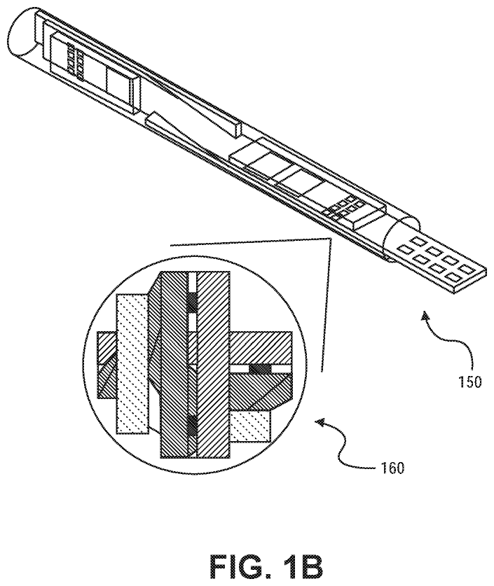

FIG. 1A shows an exemplary positioning instrument 100 with 3 dies disposed within the lumen (shown in FIG. 1B), in accordance with some embodiments of the disclosure provided herein. In one or more embodiments, positioning instrument 100 comprises three dies. Die 1 110, left, has an anisotropic magnetoresistance (AMR) full bridge for Z axis. Die 1 110v comprises 6 pads: Vdd, REG, V2−, V2+, IF−, IF+, and GND. Die 2 120, center, has anisotropic magnetoresistance (AMR) full bridges to measure X and Y axes and 8 pads: Vdd, REG, Vx−, Vx+, IF−, IF+, Vy−, Vy+, and GND. Die 3 130, right, has the analog front-end (AFE) to gain up the sensed signals and drive the output. Die 3 130 comprises 12 pads: Vdd, Vx−, Vx+, Vy−, Vy+, Vz−, Vz+, Voutx, Vouty, Voutz and GND.

The flip coils are driven in series and the IF-terminal is shared with GND. As the Flip coils need 150 mA, GND will be disturbed during flipping & analog front-end (AFE) cannot be used. Only 6 wires to the external world. The analog front-end (AFE)s are internally biased to set the output common mode voltage to VDD/2.

FIG. 1B depict an exemplary isometric view 150 and cross-sectional views 160 of a positioning instrument with 3 dies disposed within the lumen, in accordance with some embodiments of the disclosure provided herein. These orientations are demonstrative of putting on or more of the present embodiments into practice.

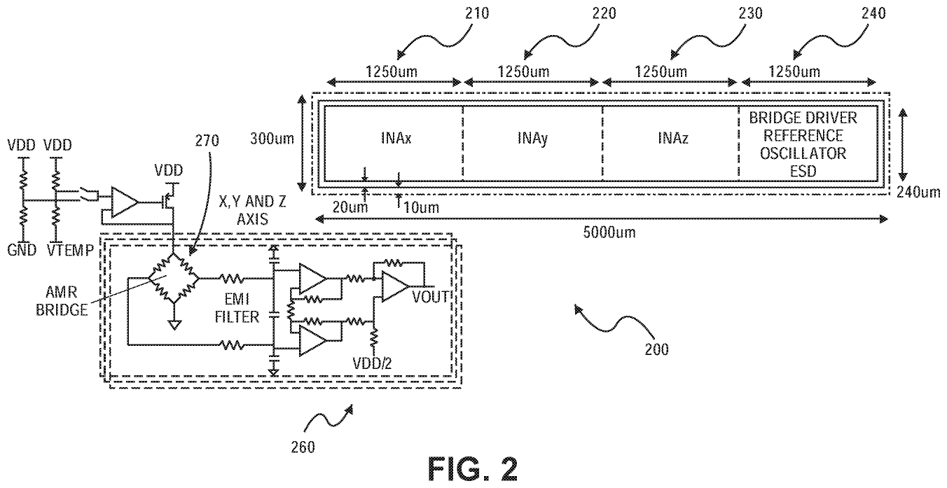

FIG. 2 shows an exemplary positioning circuit and architecture 200, in accordance with some embodiments of the disclosure provided herein. One object of the present disclosure is to measure position with 1 mm rms at a distance of ˜39 cm. This is equivalent to 6 uG for a sensing range of 1 mG to 10 G. In a preferred embodiment, sensor width is limited to 350 um. It is noted that sensor area limits total anisotropic magnetoresistance (AMR) resistance. In one or more embodiments, an anisotropic magnetoresistance (AMR) full bridge 270 of 2.5 kOhms unit resistance is used to the measure the magnetic field flux at the distal end of the catheter.

The analog front-end (AFE) 260 is a 3-amp instrumentation amplifier (INA) is used for its excellent common mode rejection ratio (CMRR) characteristic. Low noise performance is prioritized. Dimensions of analog front-end (AFE) comes to 4000 um by 350 um. Total Power dissipation (@5V) is 45 mW. Positioning circuit and architecture 200 comprises INAx 210, INAy 220, and INAz and bridge driver reference oscillator ESD 240. INAx 210 functionally receives and measure the X axis at the distal end of positioning circuit and architecture 200. Similarly, INAy 220 functionally receives and measure the Y axis near the distal end of positioning circuit and architecture 200. And, INAz 230 functionally receives and measure the Z axis at the proximal end of positioning circuit and architecture 200

The original objects of the present disclosure were 1) resolution, 2) size, 3) power. But customer feedback shows lower power dissipation is critical and achieving close to 1 mm rms resolution is also very important.

The inventors of the present disclosure recognize that optimizing the specification tradeoffs at the silicon level is important, while addressing some of the specifications at the system level. Analysis of each of the three key specifications from a system level point of view will be discussed later in the disclosure.

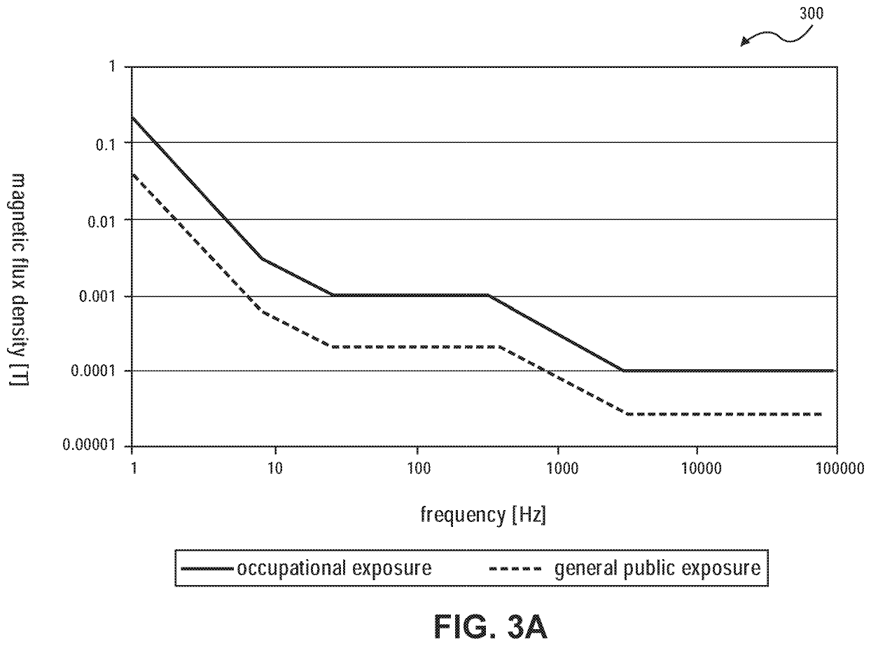

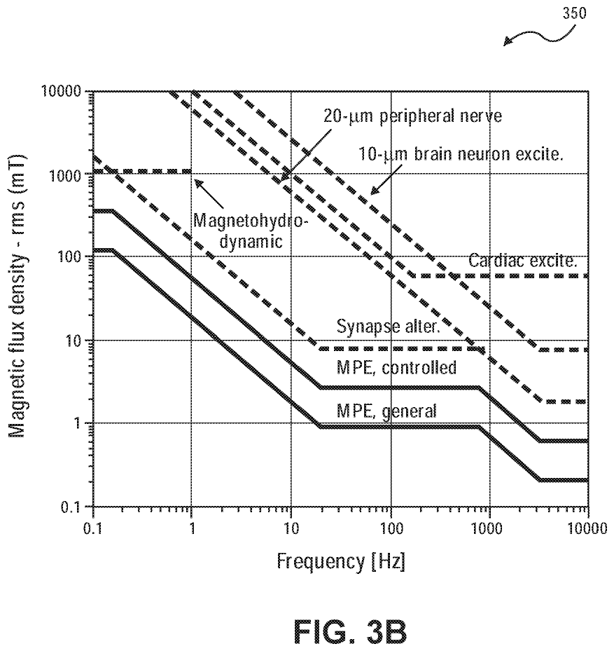

FIG. 3A graphs maximum exposure limits of magnetic flux density 300, in accordance with some embodiments of the disclosure provided herein. FIG. 3B graphs maximum exposure limits of magnetic flux density in practice, in accordance with some embodiments of the disclosure provided herein.

Practical significance of magnetobiology is conditioned by the growing level of the background electromagnetic exposure of people. Some electromagnetic fields at chronic exposures may pose a threat to human health. World Health Organization considers enhanced level of electromagnetic exposure at working places as a stress factor. Present electromagnetic safety standards, worked out by many national and international institutions, differ by tens and hundreds of times for certain EMF ranges; this situation reflects the lack of research in the area of magnetobiology and electromagnetobiology.

Standards limits maximum body exposure to 6.86 Grms˜10 Gpeak. These limits give rise to the motivation of the present disclosure. That is, a patient's body cannot exceed these levels. Yet, greater resolution et. al. which is desirable cannot be achieved without increasing the magnetic flux density. Hence, the novelty of the present disclosure which the inventors have recognized and implemented. The methodology will now be discussed in greater detail pursuant to a preferred embodiment.

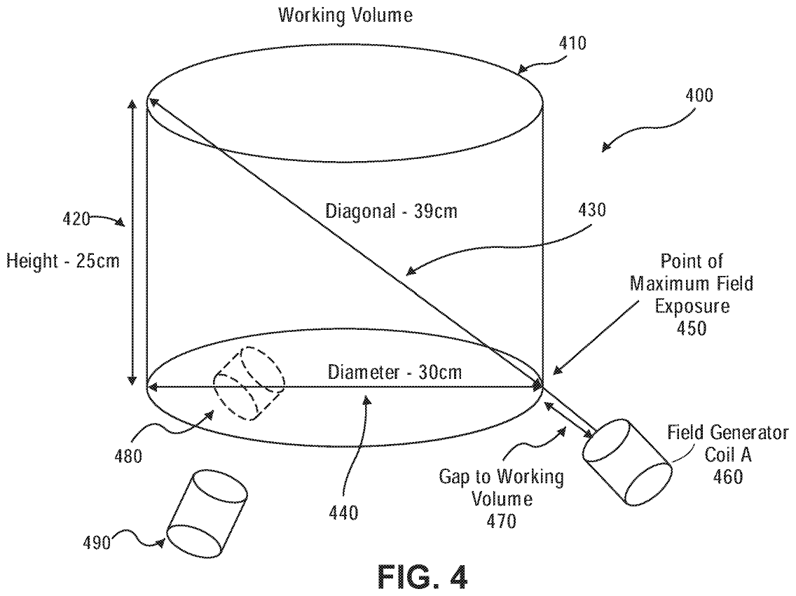

FIG. 4 illustrates an exemplary configuration of field generating coils 400, in accordance with some embodiments of the disclosure provided herein. The resolution is determined by the geometry of the field coils 460, 480, 490 and calculated as follows. The field generator coil A 460 is a certain distance (gap) 470 from the working volume. The maximum field exposure (MFE) is limited to 6.86 G rms. An object of the present disclosure is 3 mm rms resolution at a distance of 64 cm away from point of MFE. Another object of the present invention is 1 mm rms resolution at a distance of 39 cm away from point of MFE.

Working volume 410 is a function of diagonal 430, diameter 440, and height 420. To ease the resolution-power tradeoff in the catheter, the magnetic field strength needs to be increased at the catheter. At the same time, we cannot expose the human body to >6.86 Grms. Magnetic flux is the product of the average magnetic field strength and the perpendicular surface area. Flux inside the solenoid equals the flux outside.

$${∮}_{outside}^{} B dA = {∮}_{inside}^{} B dA = {μ}_{0} NI$$

For a point source field generator, the field is uniformly distributed over the surface area of a semi-sphere of a radius ‘r’

$$B = \frac{{μ}_{0} I}{2 π {r}^{2}} {}^{*} Assume N = 1 for simplicity throughout$$

Magnetic field at a point is linearly proportional to the current I in the field generator but inversely proportional to the square of the distance ‘r’ from the field generator.

The inventors of the present disclosure recognize that it is possible to limit MFE to 6.86 Grms while increasing the field strength equivalent to 1 mm rms resolution at a distance of 39 cm. Resolution options will now be discussed.

Case 1: Place the field generator 0.5 cm away from the point of maximum field exposure. To be just below the maximum exposure requirement:

$$6.86 Grms = \frac{{μ}_{0} I}{2 π {r}^{2}} where r = 0.5 cm$$

$$= > I = 86 mArms$$

Magnetic Field Strength at a distance of 39 cm from point of MFE:

$$B = \frac{{μ}_{0} I}{2 π {r}^{2}} ( where r = 39.5 cm ) = 1.09 mGrms$$

Case 2: Place the field generator 1.2 cm away from the point of maximum field exposure. This is to be just below maximum exposure requirement:

$$6.86 Grms = \frac{{μ}_{0} I}{2 π {r}^{2}} where r = 1.2 cm$$

$$= > I = 494 mArms$$

Magnetic Field Strength at a distance of 39 cm from point of MFE:

$$B = \frac{{μ}_{0} I}{2 π {r}^{2}} ( where r = 40.2 cm ) = 6.1 mGrms$$

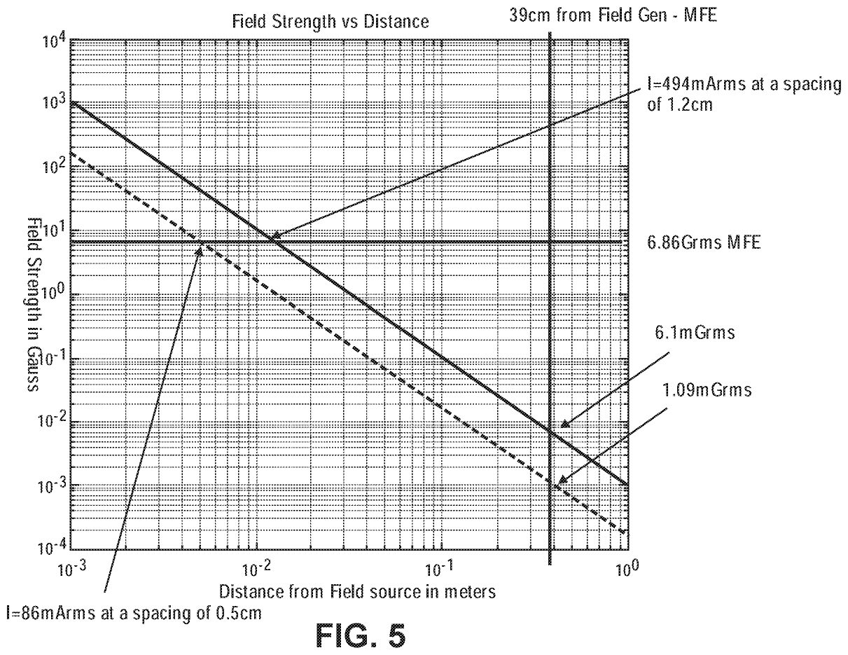

FIG. 5 depicts an exemplary chart demonstrating distal field strengths at two different field generator positions, in accordance with some embodiments of the disclosure provided herein. While both Case 1 and Case 2 have the same maximum field exposure (MFE), the magnetic field strength at the maximum distance is 5.6 times larger for Case 2.

As the distance increases the rate of reduction in magnetic field decreases.

$$\frac{d B}{dr} = - 2 \frac{{B}_{r}}{r}$$

Higher current increases the magnetic field strength everywhere. Placing the field generator, a little further away (0.5 cm→1.2 cm) reduces MFE to meet the International Electrotechnical Commission requirements.

Resolution calculation at 39 cm away from MFE is will now be discussed in greater detail.

Case 1: Place the field generator 0.5 cm away from the point of MFE with 1=86 Arms. Magnetic field strength resolution at 1 mm spacing at a distance of 39 cm from MFE point.

$${B}_{diff} = \frac{{μ}_{0} I}{2 π} \times [ \frac{1}{{( 0.395 )}^{2}} - \frac{1}{{( 0.396 )}^{2}} ]$$

This matches well with BSC's original request of 6 uGrms resolution.

Case 2: Place the field generator 1.2 cm away from the point of MFE with I=494 mArms. Magnetic field strength resolution at 1 mm spacing at a distance of 39 cm from MFE point.

$${B}_{d i f f} = \frac{{μ}_{0} I}{2 π} \times [ \frac{1}{{( 0.400 )}^{2}} - \frac{3}{{( 0 \text{.401} )}^{2}} ]$$

Resolution calculation at 64 cm away from MFE is will now be discussed and calculated.

Case 1: Place the field generator 0.5 cm away from the point of MFE with I=86 Arms. Magnetic field strength resolution at 3 mm spacing at a distance of 64 cm from MFE point.

$${B}_{d i f f} = \frac{{μ}_{0} I}{2 π} \times [ \frac{1}{{( 0.645 )}^{2}} - \frac{1}{{( 0.6 \; 48 )}^{2}} ]$$

Case 2: Place the field generator 1 cm away from the point of MFE with I=343 mArms. Magnetic field strength resolution at 3 mm spacing at a distance of 64 cm from MFE point.

$${B}_{diff} = \frac{{μ}_{0} I}{2 π} \times [ \frac{1}{{( 0.650 )}^{2}} - \frac{1}{{( 0.653 )}^{2}} ]$$

One skilled in the art can appreciate that with a higher field resolution, both power and area of the catheter can be reduced.

A system level solution to increase the required magnetic resolution will now described, according to one or more embodiments. It should be noted that the height of the field generator module increases, as a result. Also, the power of the field generator module increases but it is better to burn power in the field generators than in the catheter. Both the current and number of windings can be used to increase the field strength of the field generators.

The original proposal from the inventors provided the power & area required to meet the original resolution specification of 1 mm rms (6 uGrms) at 39 cm away. Customer feedback was that the power and aspect ratio/area are too high. Moving the limitation from the catheter to the field generators is essential for the catheter to meet its power and area targets while achieving 1 mm rms resolution.

The minimum field strength resolution can be increased from 5.6 uGrms to 31 uGrms by using field generators with 5.5× higher current or more windings. The maximum field exposure limit of 6.68 Grms is maintained by placing these stronger field generators further away from MFE.

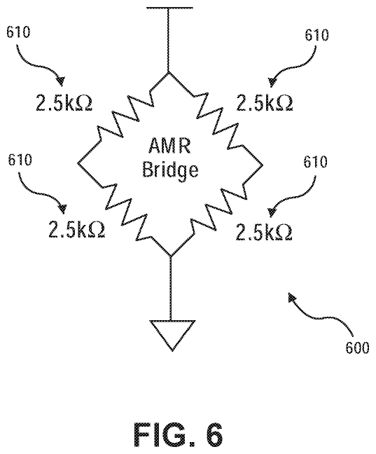

FIG. 6 is an exemplary full anisotropic magnetoresistance (AMR) bridge 600 comprising resistors 610, in accordance with some embodiments of the disclosure provided herein. Signal, Power & Noise tradeoffs will now be discussed in the context of the anisotropic magnetoresistance (AMR) bridge 600.

Dynamic Range is the ratio of Signal to Noise is given as follows:

$$D R = \sqrt{\frac{Signal Power}{Noise Power}} = \sqrt{\frac{{k}_{1} {Vdd}^{2}}{{k}_{2} R}} = {k}_{3} \frac{Vdd}{\sqrt{R}}$$

Power Dissipation is equal to Vdd2/R. Doubling the resistor, reduces power by 6 dB (½) but decreases DR by 3 dB (√2). Doubling the Vdd, increases power by 12 dB (4×) but increases DR by 6 dB (2×). So, to minimize power, a large anisotropic magnetoresistance (AMR) resistor is used.

In one or more embodiments, the limitation is area: each axis only gets 600 um×350 um die area. anisotropic magnetoresistance (AMR) resistance scales linearly with area. Original proposal was to use an anisotropic magnetoresistance (AMR) resistor full bridge with unit resistor of 2.5 kΩ. But power dissipation was 10 mW per bridge, as discussed previously which is undesirable.

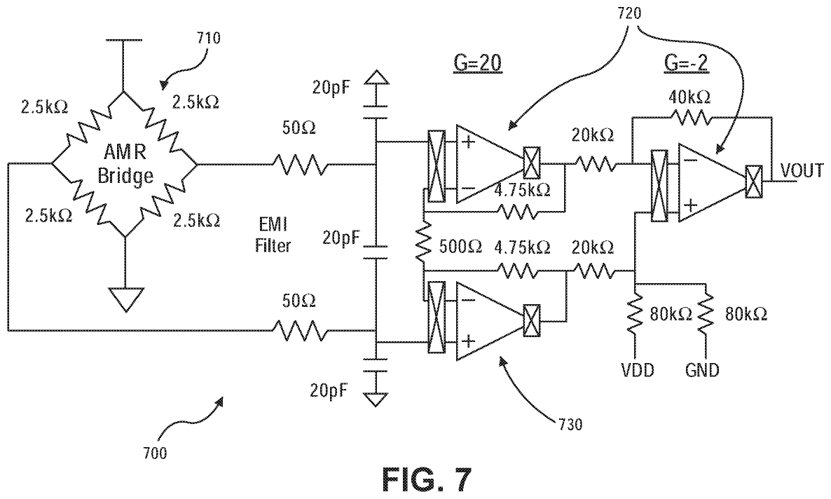

FIG. 7 demonstrates an exemplary anisotropic magnetoresistance (AMR) bridge 710 and analog front-end (AFE) 700, in accordance with some embodiments of the disclosure provided herein. The original analog front-end (AFE) proposed was a traditional 3-amp INA as we want excellent common mode rejection ratio (CMRR) and high input impedance so that the input diff voltage is not corrupted. But this architecture has 3 amplifiers 720, 730 dues to which the power dissipation of each analog front-end (AFE) is 5 mW (˜1.66 mW×3).

The main reasons for two input stage amplifiers is high input impedance for the anisotropic magnetoresistance (AMR) bridge 710 & high common mode rejection ratio (CMRR). However, total power dissipation of original analog front-end (AFE) proposal was 45 mW. The problem that arises with 45 mW is that this produces a lot of heat within a patient due to the small surface area of the catheter.

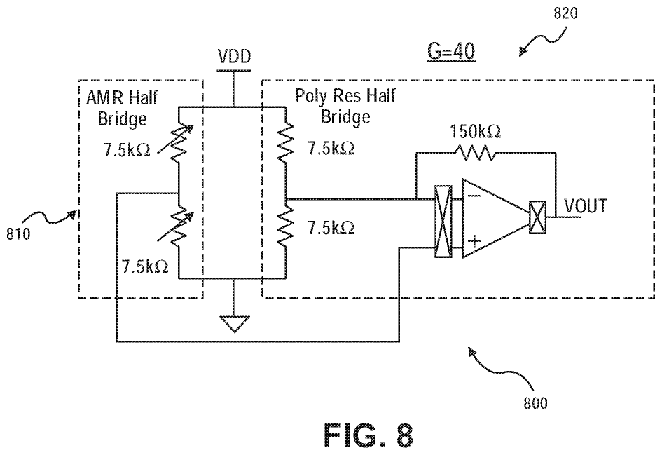

FIG. 8 demonstrates an exemplary anisotropic magnetoresistance (AMR) half bridge 810 with electrical communication with a poly res half bridge 820 disposed on the analog front-end (AFE) 800, in accordance with some embodiments of the disclosure provided herein. If an anisotropic magnetoresistance (AMR) half bridge 810 is used, just one amplifier is sufficient. Also, with an anisotropic magnetoresistance (AMR) half bridge 810, the power dissipation can be significantly reduced as the resistor can be maximized for the given anisotropic magnetoresistance (AMR) sensor die area. One of the inventors calculated 7.5 kW. But the main flaw of a half bridge is no CMRR/PSRR (common mode/power supply rejection ratios).

However, this is solved by making a second half bridge from poly resistors. A half bridge of anisotropic magnetoresistance (AMR) resistors and a half bridge of poly resistors gives a full bridge with good CMRR/PSRR. Half the signal sensitivity is lost because there is a half bridge of anisotropic magnetoresistance (AMR) resistors, but a 66% reduction in bridge power & a 66% reduction in amplifier power dissipation is achieved.

Thus, a new analog front end is proposed. The poly resistor half bridge acts to balance the bridge for good common mode rejection ratio (CMRR). The poly resistor also acts as the input resistor for the gain resistor to gain up the differential signal. The poly resistor bridge is placed on the analog front-end (AFE) die due to which the area of the anisotropic magnetoresistance (AMR) Half bridge is maximized to maximize the resistance.

The transconductance (TC) mismatch between the anisotropic magnetoresistance (AMR) and Poly resistors don't matter as we are processing the half bridge voltages. Offset voltage of the two half bridges is gained up by the amplifier. The amplifier is chopped at 100 kHz to remove 1/f noise away from the 500 Hz-5 kHz in-band.

New analog front-end (AFE) with respect to CMRR/PSRR is described as follows. The main source of common mode noise is the supply and ground signals as the bridge is integrated with the analog front-end (AFE). As analog front-end (AFE) is closely integrated with the bridge, other common mode interferers are eliminated.

DC CMRR is not important in the application as the band of interest is 500 Hz to 5 kHz. Gain of interference signals from supply/gnd is %. Gain of differential signal is 40. So, power supply rejection ratio (CMRR) in this configuration is ˜38 dB. For a 3 Amp INA, the power supply rejection ratio (CMRR) will be >80 dB in the band of interest but area is too large. Existing solution has passive power supply rejection ratio (CMRR) as the analog front-end (AFE) is not integrated into the catheter.

In one or more embodiments, the Signal & Noise effects of the new analog front-end (AFE) are as follows. The AMR half bridge thermal noise density is 8 nV/VHz. The poly resistor half bridge thermal noise density is 8 nV/VHz. The amplifier thermal noise density is 6.5 nV/VHz. The amplifier 1/f noise is chopped away from in-band. Total analog front-end (AFE) input referred noise density is 13 nV/VHz. Total analog front-end (AFE) integrated noise for 40 Hz BW is 82.5 nVrms. The AMR half bridge sensitivity is 2.5 mV/G. So, the system minimum resolution is 33 uGrms.

In one or more embodiments, the power and area of the new analog front-end (AFE) are as follows. The AMR half bridge power dissipation is (5V) 2/15 kΩ=1.66 mW. The poly resistor half bridge power dissipation is (5V) 2/15 kf=1.66 mW. The amplifier power dissipation is 1.66 mW. Power dissipation for one axis is 5 mW. Total power dissipation for all 3 axes is 15 mW. The AMR sensor Z axis die will be 600 um×350 um. The AMR sensor X and Y axis die will be 1200 um×350 um. The analog front-end (AFE) die will be 2000 um×350 um as the number of amplifier has reduced significantly.

In one or more embodiments, the new analog front-end (AFE) results in the following. The new analog front-end (AFE) achieves a minimum resolution of 33 uGrms. This is mainly determined by the resistance of the AMR resistors. So, further reduction is not possible unless we burn more power. As previously describe, by using field generators with 5.5× larger current (494 mA vs 86 mA . . . or more windings) and 2.4× wider gap (1.2 cm vs 0.5 cm), 31 uGrms is necessary to meet 1 mm rms. 1 mm rms resolution can be achieved with the stronger field generators. Total power dissipation in catheter is 15 mW. Total length for sensor die+AFE<=4 mm.

To summarize, the present state of the art is hitting pretty fundamental physics limitation for the catheter from a resolution, power & area point of view. Hence, the inventors have disclosed a system level solution to ease the limitations.

Increasing the magnetic field strength of the field generators is one approach. It is easier to ease the limitations in the field generators than the small, low power catheter.

Placing the stronger field generators further away from point of maximum field exposure (MFE) enables us to meet the Standards requirements for MFE.

New analog front-end (AFE) minimizes power dissipation by using an anisotropic magnetoresistance (AMR) half bridge with a poly resistor half bridge and using a single amplifier instead of a 3-amp INA. This enables us to meet 1 mm rms resolution, 15 mW power dissipation and 4 mm total catheter length for anisotropic magnetoresistance (AMR) sensor dies & analog front-end (AFE) die combined.

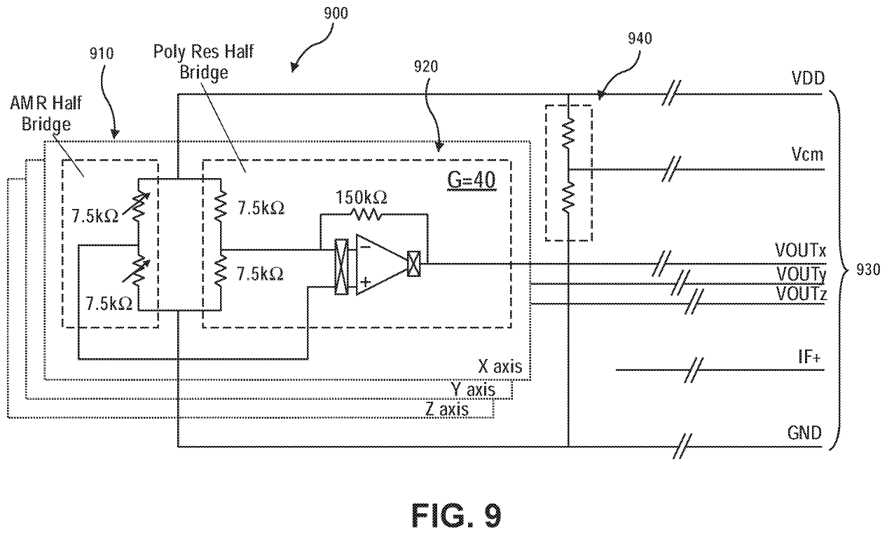

FIG. 9 illustrates an exemplary an exemplary anisotropic magnetoresistance (AMR) half bridge 910 with electrical communication with a poly res half bridge 920 disposed on the analog front-end (AFE) 900 and an additional pin to output common mode catheter voltage, in accordance with some embodiments of the disclosure provided herein. The present embodiment improves power supply rejection ratio (CMRR) and will now be described.

An extra pin is used to sense Vcm 940 at the catheter, which yields first order cancellation of power supply/gnd interference. Gain of power supply/gnd interference signals from bridge to amplifier output is %. Gain of differential signals from bridge to amplifier output is 40. Gain of interference signals from supply/gnd to Vcm is %.

The Vcm divider 940 can be shared between all 3 axes. Output signals will now be differential measurements between Vout and Vcm. Additionally, interference on the Vout signals is also now common mode. Cost: Extra pin (Vcm) 940, 1.66 mW power dissipation and 10% increase in noise. Output 930 increase to 7 as opposed to 6 in one of the previous embodiments.

In some embodiments, the field generators are solenoids. However, numerous other magnetic field generators are not outside the scope of the present disclosure. These include electromagnets, permanent magnets or any combination thereof, all of which are known in the art.

In a preferred embodiment, anisotropic magnetoresistors (AMR) are use are used. Nonetheless, other magnetoresistive elements, such as, Tunnel magnetoresistance (TMR) and Giant magnetoresistance (GMR) sensors, are not outside the scope of the any of the disclosed embodiments.

The key specifications for the magnetic position system are a resolution of 1 mm rms, very low power dissipation and 4 mm×350 um die area for all 3 axes. The resolution requirement of 1 mm rms was equivalent to measuring 6 uGrms. Original approaches for this design included a full anisotropic magnetoresistance (AMR) sensor bridge coupled with a 3-amp INA. The noise of the system was reduced to meet the 1 mm rms resolution requirement.

This yielded a power dissipation of 45 mW and area close to 8 mm×350 um. In addition, the number of wires to the catheter is limited to <=6. Customer conveyed that this solution was not good enough for their requirements. Customer's current system uses solenoid coil to measure magnetic fields. It is low power and noise but size is too large. They are currently at 0.8 mm diameter and want to get to 0.5 mm diameter with our solution.

In one or more embodiments, a full anisotropic magnetoresistance (AMR) bridge sensitivity is 1 mV/V per Gauss. The signal range is from 1 mG to 10 G. The minimum resolution to be measured is 6 uGrms. So, with a 5V bridge supply, the minimum resolution required is 30 nVrms. The measurement bandwidth is 40 Hz. So, this is equivalent to 4.7 nV/rt(Hz) for bridge+Amp. Power dissipation per channel has to be less than 5 mW (bridge and INA). Finally, the sensors take up 600 um×350 um for each axis. So, the analog front-end (AFE) area for all 3 axes is 2000 um×350 um. The area and power requirements make this very challenging. Frequency of interest is from 500 Hz to 5 kHz

To summarize some of the previous embodiments. A catheter with very limited in area and power dissipation and the 6 uGrms field requirement is too low. So, an increase in the magnetic field strength is needed at the sensing point. But, there is a system requirement that the patient cannot be exposed to more than 6.86 Grms field strength.

Increasing the field strength at the sensing point while not exposing the patient to the maximum field strength standard is contemplated. During analysis described in attached figures, field strength can be increased by increasing magnetic flux by increasing current or number of windings in the field generators.

To still meet the standards, field generators are disposed further away. As the rate of reduction in field strength decreases with distance, this technique allows us to have stronger magnetic field strength at the point of interest while still meeting the standards for maximum field exposure. This enables a relaxation of the field strength equivalent for 1 mm rms resolution from 6 uGrms to 31 uGrms.

In one or more embodiments, the main constraint in the catheter design is area. A maximum area of 600 um×350 um is devoted for the full anisotropic magnetoresistance (AMR) sensor for each axis. If a full bridge is built in this area, the maximum unit resistance is 2.5 kOhms. AT 5V bridge supply, this give 10 mW in power dissipation for each axis. Once a 3 Amp INA is included, the total power dissipation for all 3 axes is 45 mW. The goal is to get to 15 mW.

The insight here is that supporting neither a full bridge nor a 3-amp INA is feasible in this area limitation. So, a half bridge of anisotropic magnetoresistance (AMR) sensors can be combined with a half bridge of poly resistor sensors. Now, the anisotropic magnetoresistance (AMR) half bridge is total of 15 kOhms in the same area. So, half bridge power dissipation is 1.66 mW. A signal sensitivity is reduced by half, but the previous embodiment yielding more magnetic flux density achieves more signal.

In one or more embodiments, the anisotropic magnetoresistance (AMR) half bridge allows the use of just a single amplifier. The only resistor half bridge enables to achieve a decent CMRR (40 dB) while also acting in conjunction with the gain resistor to set the differential gain. The single amp is chopped with a ripple suppression loop to remove chopping ripple. This eliminates 1/f noise. This also enables to make the analog front-end (AFE) die just 2000 um by 350 um for all 3 axes.

The result of the aforementioned embodiments in the summary is that meets 3 main specifications in practice: 1 mm rms resolution; 15 mW total power dissipation; and 4 mm of catheter length to deliver a break through solution in the minimally invasive surgical instrumentation market with anisotropic magnetoresistance (AMR) sensors.

Existing solutions from other medical instrument makers uses solenoid coil based magnetic position sensing. The major challenge with this approach is the size of the catheter. The smallest size for existing solution is 800 um diameter. The solution disclosed based on anisotropic magnetoresistance (AMR) sensors will deliver a 500 um diameter catheter positioning solution delivering a breakthrough in the application space.

The area, power and resolution requirements make this a very restrictive problem.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein.

Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments may be implemented in any of numerous ways. One or more aspects and embodiments of the present application involving the performance of processes or methods may utilize program instructions executable by a device (e.g., a computer, a processor, or other device) to perform, or control performance of, the processes or methods.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above.

The computer readable medium or media may be transportable, such that the program or programs stored thereon may be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that may be employed to program a computer or other processor to implement various aspects as described above. Additionally, it should be appreciated that according to one aspect, one or more computer programs that when executed perform methods of the present application need not reside on a single computer or processor but may be distributed in a modular fashion among a number of different computers or processors to implement various aspects of the present application.

Computer-executable instructions may be in many forms, such as program modules, executed by one or more computers or other devices. Generally, program modules include routines, programs, objects, components, data structures, etc. that performs particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a personal digital assistant (PDA), a smart phone, a mobile phone, an iPad, or any other suitable portable or fixed electronic device.

Also, a computer may have one or more input and output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Elements other than those specifically identified by the “and/or” clause may optionally be present, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “between” is to be inclusive unless indicated otherwise. For example, “between A and B” includes A and B unless indicated otherwise.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1-20. (canceled)

21. A catheter, comprising:
for each of a first axis, a second axis, and a third axis:
a bridge circuit comprising:
a magneto resistive half bridge comprising a first magneto resistance and a second magneto resistance in series between a voltage supply and ground;
a poly resistive half bridge comprising a first poly resistance and a second poly resistance in series between the voltage supply and the ground; and
a single stage operational amplifier comprising a differential input,
wherein a first part of the differential input is in electrical communication with a midpoint between the first magneto resistance and the second magneto resistance, and
wherein a second part of the differential input is in electrical communication with a midpoint between the first poly resistance and the second poly resistance.

22. The catheter of claim 21, further comprising:
a circuit board; a first die; a second die; and a third die,
wherein:
each die is on the circuit board;
the first axis magneto resistive first half bridge is on the first die;
the second axis magneto resistive first half bridge and third axis magneto resistive first half bridge are on the second die; and
each poly resistive second half bridge and each single stage operation amplifier is on the third die.

23. The catheter of claim 22, wherein the dies are in series on the circuit board along a longitudinal axis of the catheter.

24. The catheter of claim 21, wherein:
the first magneto resistance and the second magneto resistance are equal resistances;
the first poly resistance and the second poly resistance are equal resistances;
the first part of the differential input is a positive input;
the second part of the differential input is a negative input; and
the single stage operational amplifier comprises a negative feedback resistance.

25. The catheter of claim 21, wherein the catheter comprises no flip coils.

26. The catheter of claim 21, further comprising:
a first common mode resistor and a second common mode resistor in series between the voltage supply and the ground; and
an amplifier output sense pin per single stage amplifier output;
a common mode voltage pin in communication with a midpoint between the first common mode resistor and the second common mode resistor.

27. A method of operating a catheter, comprising:
providing a catheter comprising:
for each of a first axis, a second axis, and a third axis:
a bridge circuit comprising: a magneto resistive half bridge comprising a first magneto resistance and a second magneto resistance in series between a voltage supply and ground; a poly resistive half bridge comprising a first poly resistance and a second poly resistance in series between the voltage supply and the ground;
a single stage operational amplifier comprising a differential input, wherein a first part of the differential input is in electrical communication with a midpoint between the first magneto resistance and the second magneto resistance, and wherein a second part of the differential input is in electrical communication with a midpoint between the first poly resistance and the second poly resistance; and
an amplifier output sense pin in electrical communication with an output of the amplifier;
inserting the catheter into a working volume of a set of magnetic field generators;
measuring a voltage of each amplifier output sense pin; and
determining a position of the catheter in the working volume based the measured voltage.

28. The method of claim 27, wherein:
the catheter further comprises: a first common mode resistor and a second common mode resistor in series between the voltage supply and the ground, and a common mode voltage pin in communication with a midpoint between the first common mode resistor and the second common mode resistor; and
measuring a voltage of each amplifier output sense pin comprises measuring a voltage between each amplifier output sense pin and the common mode voltage pin.

29. The method of claim 27, wherein
the catheter further comprises a circuit board; a first die; a second die; and a third die,
wherein:
each die is on the circuit board;
the first axis magneto resistive first half bridge is on the first die;
the second axis magneto resistive first half bridge and third axis magneto resistive first half bridge are on the second die; and
each poly resistive second half bridge and each single stage operation amplifier is on the third die.

30. The method of claim 29, wherein the dies are in series on the circuit board along a longitudinal axis of the catheter.

31. The method of claim 27, wherein:
the first magneto resistance and the second magneto resistance are equal resistances;
the first poly resistance and the second poly resistance are equal resistances;
the first part of the differential input is a positive input;
the second part of the differential input is a negative input; and
the single stage operational amplifier comprises a negative feedback resistance.
